(19) United States
(12) Patent Application Publication
(10) Pub. No.: US 2004/0249302 A1
(43) Pub. Date: Dec. 9, 2004

(54) Methods and systems for processing of brain signals

(75) Inventors: John P. Donoghue, Providence, RI (US); Nicholas George Hatsopoulos, Chicago, IL (US); Mijail Serruya, Providence, RI (US)

Correspondence Address: Finnegan, Henderson, Farabow,, Garrett & Dunner, L.L.P., 1300 I Street, N.W., Washington, DC, 20005-3315, US

(73) Assignee: Cyberkinetics, Inc.

(21) Appl. No.: 10/456,490

(22) Filed: Jun. 9, 2003

(51) Int. Cl. A61B 5/04

(52) U.S. Cl. 600/544

(57) ABSTRACT

System and methods consistent with the present invention decode brain signals. The system includes a receiver for receiving an input signal representing multiple individual neuron signals, and a frequency filter for separating the multiple neuron signals from the received input neural signal. A rectifier full wave rectifies the filtered neural signal and an integrator integrates the rectified neural signal to obtain an envelope of the rectified neural signal. As a result of this processing, sample values of the neural signal envelope represent neurological activity.

### FIELD OF THE INVENTION

The present invention pertains to processing of brain signals, and, more particularly, to methods and systems for processing brain signals representing multiple neuron signals.

### BACKGROUND OF THE INVENTION

Recent advances in neurophysiology have allowed researchers to study the electrical activity of highly localized groups of neurons with high temporal accuracy and in specific locations in the brain. These advances create the possibility for brain-computer interfaces allowing an amputee to control a prosthetic limb in much the same way that the amputee would control a natural limb. Although noninvasive sensors, such as multichannel electroencephalogram (EEG) sensors placed on the surface of a person's skin, have been used as simple brain-computer interfaces, they do not currently offer the temporal or spatial resolution needed for prosthetic control. Such noninvasive sensors can detect only mass fluctuations of neuron activity that have been attenuated by the intervening bone and tissue. As a result, these types of brain-computer interfaces can derive only simple forms of information from the neuron activity. They also operate very slowly because the mass neuron signal activity only modulates at very low rates, requiring more processing time.

More advanced brain-computer interfaces use sensing electrodes placed directly in contact with the brain to detect neuron activity. These electrodes, which may comprise a micro-wire or hatpin-like electrode, each form a recording channel that directly detects the electrical impulse signal from all of the neurons in the electrode's vicinity. Further signal processing then isolates the individual neuron signals, each of which comprises a series of electrical spikes reflecting information correlated to a respective function (e.g., a particular movement of a particular limb). The brain encodes this information according to the frequency or firing rate of the spikes. By collecting the firing rates of a number of individual neuron signals detected via a number of recording channels, a brain-computer interface can derive control signals to control a neural prosthetic device.

While such control signals provide about a hundred times more information than noninvasive sensors, the processing of directly detected single neuron signals is much more computationally intensive than the noninvasive brain-computer interfaces. For instance, a data acquisition system typically performs the following functions to derive a control signal: (1) amplify the electrical impulse signals from a number of recording channels; (2) bandpass filter the amplified signal of each channel to remove unwanted noise; (3) digitize the filtered signals; (4) detect and isolate the spikes of individual neuron signals forming each digitized signal by applying threshold or waveform detection functions; (5) store the time each detected signal spike occurs and the digital waveform data associated with each detected spike; and (6) decode the neuron activity by applying recognition logic to associate the spike times of a particular neuron or groups of neurons with various behavioral events (such as a desired movement of a limb).

The above calculations produce a large amount of data and require a large amount of processing time. For example, the filtered neuron signals must be digitized at a high rate, such as 30 kHz. When using an electrode array having a large number of electrodes or recording channels, this produces a large amount of data. If 100 channels record neuron activity, the information rate in the above example may then be 30 kilo-samples×100×14 bits=4 Mbits/sec. Such information signals can only be rapidly processed by using fast processing devices that often require significant power and generate considerable heat.

There are several significant obstacles to using such processing devices with direct brain-computer interfaces. Most notably, since the information rate for a larger number of channels is too high for current methods of wireless transmission, these interfaces require percutaneous connectors to physically connect the electrode array with an external processor. These connectors present a source of infection that potentially limits the useful life of these systems. In addition, the cables themselves also present additional problems in the design of a prosthesis that must continue to function over many years and not interfere with the patient's daily life. Finally, the relatively long cables present a source of electrical interference.

Accordingly, there is a need for a brain-computer interface that may process multiple neuron signals using a low bandwidth or reduced information rate. Further, there is a need for such interfaces to allow for an internal processing device that may have low heat generation, low power consumption, and/or decreased processor complexity.

### SUMMARY OF THE INVENTION

According to a first aspect of the invention, a system is provided for processing neural signals. The system includes a receiver for receiving an input signal representing multiple individual neuron signals, and a frequency filter for separating the multiple neuron signals from the received input neural signal. A rectifier full wave rectifies the filtered neural signal and an integrator integrates the rectified neural signal to obtain an envelope of the rectified neural signal. As a result of this processing, sample values of the neural signal envelope represent neurological activity.

According to a second aspect of the invention, a method is provided for processing neural signals. The method includes receiving an input neural signal representing multiple individual neuron signals and filtering the received input neural signal to separate the multiple neuron signals from the received input signal. The filtered neural signal is then full wave rectified and the rectified neural signal is then integrated to obtain an envelope of the rectified neural signal. As a result of this processing, sample values of the neural signal envelope represent neurological activity.

Both the foregoing general description and the following detailed description are exemplary and are intended to provide further explanation of the embodiments of the invention as claimed.

### BRIEF DESCRIPTION OF THE DRAWINGS

The accompanying drawings, which are incorporated in and constitute a part of this specification, illustrate various embodiments of the present invention, and, together with the description, serve to explain the principles of the invention. In the drawings:

FIG. 1 is a diagram illustrating an exemplary brain-computer interface system consistent with an embodiment of the present invention;

FIG. 2 is a block diagram of a neuron signal decoding system consistent with an embodiment of the present invention; and

FIGS. 3A-E illustrate signal waveforms of the neuron signal processed by the neuron signal decoding system.

### DESCRIPTION OF THE EMBODIMENTS

Reference will now be made in detail to exemplary embodiments of the invention, which are illustrated in the accompanying drawings. Wherever possible, the same reference numbers will be used throughout the drawings to refer to the same or like parts.

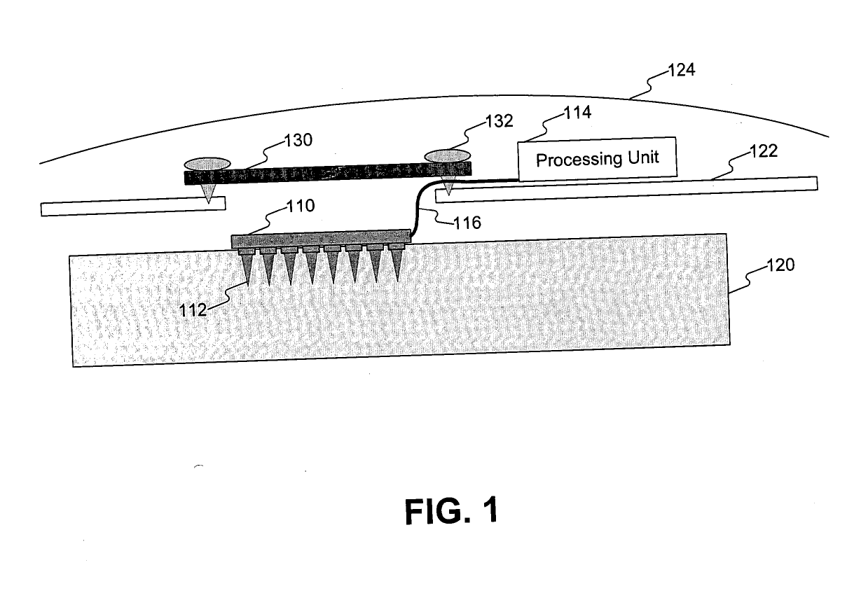

FIG. 1 generally illustrates a brain-computer interface system consistent with an exemplary embodiment of the present invention. As shown in FIG. 1, the system includes an electrode array 110 inserted into a patient's cerebral cortex 120 through an opening in the skull 122. Array 110 may include one or more electrodes 112 for detecting electrical brain signals or impulses from within or on the surface of cortex 120. Thus, for example, electrodes 112 may be inserted into the motor cortex to record neuron signals associated with a patient's motor control, or may be inserted into the visual cortex to record neuron signals associated with the patient's visual functions. Each electrode 112 of array 110 may be connected to a multi-unit activity (MUA) processor 114 via a cable 116. In exemplary embodiments, array 110 may then be sealed in skull 122. While FIG. 1 illustrates, for exemplary purposes, covering array 110 with a protective plate 130 secured to skull 122 by screws 132, the present invention may use any of a number of known protective plates and methods for attaching the same to a patient's skull. Moreover, the present invention may also be used without a protective plate by, for example, inserting array 110 via a probe.

Electrode array 110 serves as the sensor for the brain implant system. As shown in FIG. 1, each electrode 112 may extend into cortex 120 to detect the electrical neural signals generated from the neurons located in proximity to the electrode's placement within the brain. Neurons may generate such signals when, for example, the brain instructs a particular limb to move in a particular way, generates cognitive intent, or receives a sensory input. The electrical neural signals detected by each electrode are referred to herein as MUA (multiunit activity) signals since these neural signals reflect the electrical activity of multiple neural unit signals.

While FIG. 1 shows array 110 as having eight electrodes 112 for detecting MUA signals, array 110 may have any number of electrodes that may be arranged in a variety of ways. For example, array 110 may comprise a two-dimensional array of electrodes or just one electrode. Moreover, any type of electrode for sensing MUA signals may be used with the present invention. The electrodes may thus have a variety of forms, shapes, and sizes and may be of types other than that shown in FIG. 1. For instance, the invention may use electrodes known in the art that sense MUA signals by lying on the surface of cortex 120 rather than by extending into it.

Electrodes 112 may transfer the detected MUA signals to MUA processor unit 114 over cable 116. While FIG. 1 shows cable 116 passing out of the opening in skull 122 beneath protective plate 130 to connect to MUA processor 114, cable 116 may connect to processor 114 in a number of alternative ways. For example, cable 116 may pass through a hole in skull 122 or in plate 130. Further, cable 116 may be implemented by using any type of signal conductor, including, for example, metal wire or coaxial cable.

In systems consistent with the invention, MUA processor 114 may process either the raw MUA signals detected by electrodes 112 or the raw MUA signals after a preprocessing stage. In the later case, the raw detected MUA signals may be preprocessed to reflect a particular group of neuron signals, rather than the entire group of neuron signals reflected in the raw signal. For instance, the raw MUA signal typically reflects the multiple neuron signals detected by a respective electrode. If only a subset of those neuron signals are desired, the raw detected MUA signals may be preprocessed to filter the desired subset of neuron signals from the entire set detected. To filter the desired subset of neuron signals from each of the detected signals, a thresholding technique known to those skilled in the art may be used. Such known thresholding techniques filter desired neuron signals based on the signal amplitude level of the desired signals. Persons skilled in the art will appreciate, however, that other filtering techniques may be used to obtain the desired MUA signals.

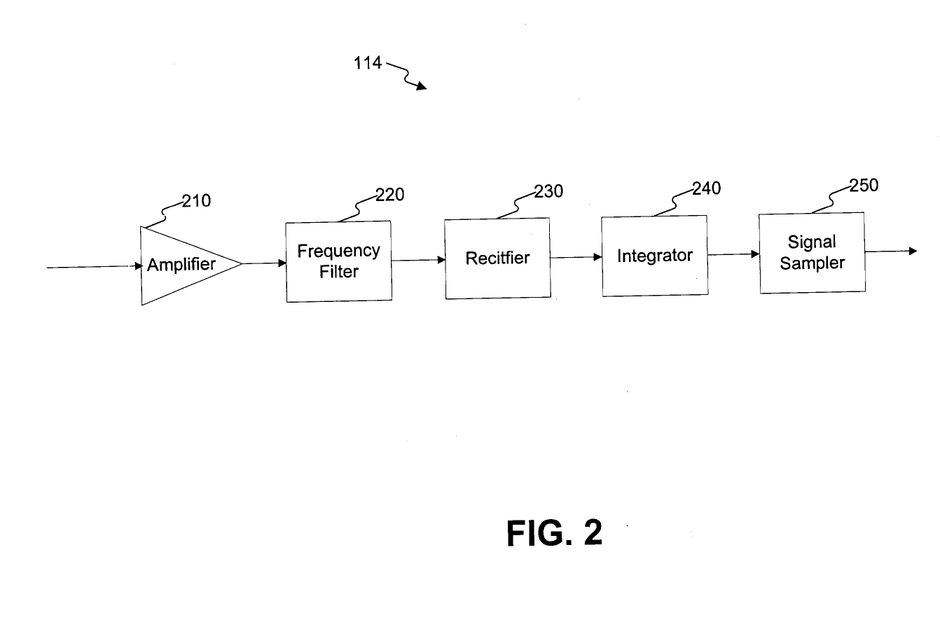

MUA processor 114 may then further process the MUA signals to extract neural information that it may then transmit to an external control device (not shown). For example, the control device may, as known in the art, use the transmitted neural information to decode that information for control of a motorized prosthetic device, for stimulation of the brain (e.g., for providing visual sensory input), or for input into another computing system for analysis or for a variety of other purposes. While FIG. 1 shows MUA processor 114 as being located between skull 122 and skin 124, processor 114 may be located elsewhere, such as inside skull 122 or outside the patient's body, for example. MUA processor 114 is described in further detail with respect to FIG. 2.

FIG. 2 is a block diagram of MUA processor 114 consistent with an exemplary embodiment of the present invention. As shown in FIG. 2, MUA processor 114 may further include an amplifier 210, a filter 220, a rectifier 230, an integrator 240, and a sampler 250. As described above, MUA processor 114 receives and processes the MUA signal output by electrode 112 of array 110. Each received MUA signal represents the electrical activity of the neurons located in proximity of the respective electrode 112.

Amplifier 210 is a preamplifier stage that amplifies the analog MUA signal of each recording channel received from an electrode 112. Amplifier 210 may amplify the MUA signals from all electrodes 112 in array 110, or may include a set of amplifiers each of which may amplify a particular set of electrodes 112. In one exemplary embodiment, amplifier 210 may be implemented using a CMOS operational amplifier known to those skilled in the art, and may have a bandwidth of approximately 1-8 kHz and a gain of about 100. However, amplifier 210 may be implemented using, for instance, different bandwidth and gain values that are selected based on the characteristics of the electrode and the detected signal.

Filter 220 may then filter each amplified MUA signal to remove unwanted frequency noise signals. In the exemplary embodiment, filter 220 is implemented using a 300 Hz to 10 kHz bandpass filter. Further, filter 220 may be tuned to pass fixed frequency signals based on the frequencies of the type of neuron signals within the detected MUA signal. Filter 220 may also adaptively filter the amplified neural signals by adaptively adjusting the filtered frequency signals based on the frequency of the individual or collective neuron signals within the detected MUA signal.

Rectifier 230 may receive each filtered MUA signal to full wave rectify those signals. In particular, rectifier 230 may convert the filtered MUA signal to its absolute value. Integrator 240 may then integrate the rectified MUA signal to obtain the envelope of the rectified MUA signal. In an exemplary embodiment, integrator 240 may be implemented by a low pass filter having a cut off frequency of about 200 Hz. However, persons skilled in the art will readily understand that other implementations of integrator 240 are possible and may be chosen based on the frequency components of the rectified MUA signal.

Signal sampler 250 may then sample the integrated MUA signal to transform it into a digital form. In systems consistent with the present invention, sampler 250 may include an A/D converter that samples the MUA signal at a low sampling rate, such as at a sampling rate of less than 1-2 kHz. MUA processor 114 may utilize a low sampling rate because the above processing by filter 220, rectifier and integrator 240 results in a sampled MUA signal carried by a low frequency. Thus, a sampling rate of 1 kHz may even result in over sampling the MUA signal by five times the minimum sampling rate. In this way, MUA processor 114 produces an output signal having a low bandwidth or reduced information rate, while still accurately representing the detected MUA signal. MUA processor 114 can thus be implemented by using smaller components, or those operating at low sampling rates (e.g., 1 kHz versus 30 kHz), that meet heat dissipation and power consumption design criteria for a system mounted in or on the patient's body.

When multiple electrodes 112 are used to detect the MUA signals, sampler 250 may sample and multiplex those channel signals into a single data stream. While the multiplexing of the MUA signals will increase the data rate of the multiplexed signal based on the number of channels multiplexed, the increased data rate may still be well within the processing range of many known A/D converters due to the above processing operations of MUA processor 114. Further, the above processing operations which result in the low bandwidth output signal may then be used to design smaller components, with less heat production and power demands, for transmitting the output signal. In systems consistent with the invention, the output signal may be transmitted from the brain-computer interface to an external control device by using, for example, fiber optic transmission, “Bluetooth”, or IEEE 802.11 technology, or according to any other type of wireless communication standard, such as code division multiple access (CDMA), wireless application protocol (WAP), or infrared telemetry.

Sampler 250 may be implemented by using a number of signal sampling devices. In an exemplary embodiment, sampler 250 is implemented using an A/D converter, as noted above. Further, the sampling rate may be a fixed sampling rate, a sampling rate that may adapt to changes in the integrated MUA signal, or a sampling rate that may be modified by an operator. Further, while FIG. 1 shows the rectifying and integrating stages preceding the sampling stage, systems and methods consistent with the invention may include implementing the processing of rectifier 230 and integrator 240 after sampling of the filtered MUA signal by sampler 250.

The resulting signal output by sampler 250 may be represented in a number of forms. For instance, the output signal may be represented as a time/amplitude signal represented by a pulse train in which each pulse has an amplitude level corresponding to the respective sample value. The output signal may also be represented by a voltage/frequency signal in which the frequency is varied to correspond to the respective sample value.

The above processing by MUA processor 114 eliminates the need for a brain-computer interface to discriminate each individual neuron signal. In particular, by filtering, rectifying, and integrating an MUA signal representing all of the neurons in the vicinity of an electrode, processor 114 eliminates the computationally intensive process for discriminating spikes of individual neurons and/or transmitting the high frequency waveforms of individual neurons. Further, since the MUA signal processed by processor 114 reflect the same function (e.g., an intended movement of a particular limb) as that reflected by an individual neuron signal, the output of processor 114 may be accurately used to determine a motor control signal for controlling a prosthetic device.

As noted above, the digital sample values output by sampler 250 may then be transmitted to an external control device (not shown) that decodes the sample values for generating a control signal for control of a motorized prosthetic device or other devices for controlling or assisting other body parts (e.g., to provide sensory stimulation for vision). Such decoders are well known to those skilled in the art and translate the sample values, output from one or more electrode channels, into a control signal. For instance, applicable neural signal decoding processes of this type are described in: “Direct Cotrical Control of 3D Neuroprosthetic Devices,” D. M. Taylor et al., Science, Vol. 296, (Jun. 7, 2002); “Real-time Prediction of Hand Trajectory by Ensembles of Cortical Neurons in Primates,” J. Wessberg et al., Nature, Vol. 408 (Nov. 16, 2000); and “Inferring Hand Motion from Multi-Cell Recordings in Motor Cortex Using a Kalman Filter,” W. Wu et al., SAB'02-Workshop on Motor Control in Humans and Robots: On the Interplay of Real Brains and Artificial Devices, Aug. 10, 2002, Edinburgh, Scotland (UK), each of which are incorporated herein by reference in their entirety.

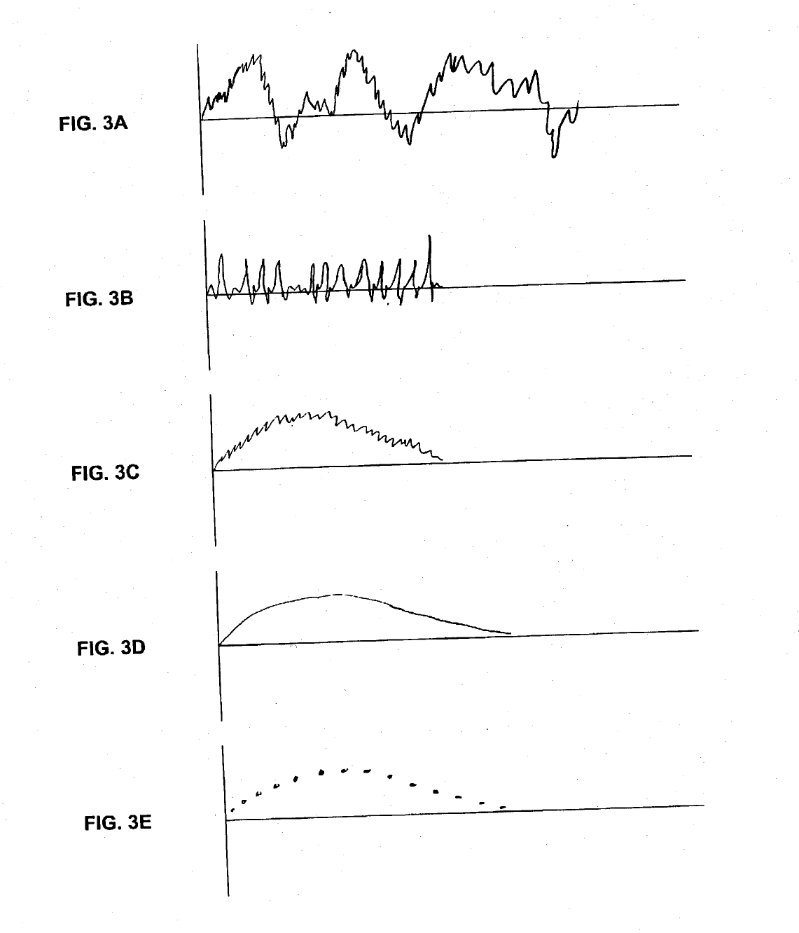

FIGS. 3A-E illustrate signal waveforms of the neuron signal processed by processing unit 114 as described above. In particular, FIG. 3A illustrates the waveform of an analog MUA signal output by an electrode 112 and received by amplifier 210. The analog neuron signal of FIG. 3A is composed of a multiple of neuron signals detected by the electrode. FIG. 3B illustrates the waveform of the analog neuron signal after being filtered by filter 220. As shown in FIG. 3B, filter 220 may remove low and/or high frequency noise signals to produce an analog MUA waveform corresponding to the summed activity of the neurons located in proximity to an electrode's placement within the brain. FIG. 3C illustrates the analog waveform of FIG. 3B after being rectified by rectifier 230. FIG. 3D then illustrates the rectified waveform of FIG. 3C after being integrated by integrator 240. As shown in FIG. 3D, the integrator obtains the envelope of the rectified signal's waveform. Finally, FIG. 3E illustrates the output of sampler 240 and shows the sampled values of the output of integrator 240. The output signal (FIG. 3E) of MUA processor 114 may then be provided to a neuron signal decoding algorithm processor, as described above.

Accordingly, methods and systems for processing neural signals in a brain-computer interface have been described above. The individual system components described herein may be specifically constructed for performing various processes and operations of the invention or they may include a general purpose computer or computing platform selectively activated or reconfigured by program code to provide the necessary functionality. The processes disclosed herein are not inherently related to any particular computer or apparatus, and may be implemented by any suitable combination of hardware, software, and/or firmware. For example, various general purpose machines may be used with programs written in accordance with the teachings of the invention, or it may be more convenient to construct a specialized apparatus or system to perform the required methods and techniques.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A system for processing neural signals, comprising:
a receiver for receiving an input neural signal representing multiple individual neuron signals sensed directly from a cerebral cortex;
a frequency filter for separating the multiple neuron signals from the received input neural signal;
a rectifier for full wave rectifying the filtered neural signal; and
an integrator for integrating the rectified neural signal to obtain an envelope of the rectified neural signal,
wherein sample values of the neural signal envelope represent neurological activity.

2. The system of claim 1, wherein the receiver further includes:
an amplifier for amplifying the received input signal.

3. The system of claim 1, wherein the frequency filter comprises a bandpass filter.

4. The system of claim 3, wherein the bandpass filter passes frequencies between 300 Hz and 10 kHz.

5. The system of claim 1, wherein the integrator comprises a low pass filter.

6. The system of claim 1, further including a sampler to obtain the sample values, and wherein the sampler samples the integrated neural signal.

7. The system of claim 1, further including a sampler to obtain the sample values, and wherein the sampler samples the filtered neural signal such that the rectifier full-wave rectifies the sampled neural signal.

8. The system of claim 1, further including a sampler to obtain the sample values wherein the sampler samples the integrated neural signal according to a low sampling rate.

9. The system of claim 8, wherein the sampling rate is less than about 2 kHz.

10. The system of claim 1, wherein the sampler further includes:
an analog-to-digital (A/D) converter for converting the neural signal into a digital signal.

11. The system of claim 10, wherein the A/D converter samples the neural signal according to a low sampling rate.

12. The system of claim 11, wherein the sampling rate is less than about 2 kHz.

13. The system of claim 1, wherein the receiver further includes:
an electrode for making electrical contact with the cerebral cortex to thereby detect the input neural signal.

14. The system of claim 13, wherein the electrode is inserted into the cerebral cortex to thereby detect neural signals below a surface of the cerebral cortex.

15. The system of claim 13, wherein the electrode is part of a two-dimensional array of electrodes.

16. A method for decoding a neuron signal, comprising:
receiving an input neural signal representing multiple individual neuron signals sensed directly from a cerebral cortex;
filtering the received input neural signal to separate the multiple neuron signals from the received input signal;
full-wave rectifying the filtered neural signal;
integrating the rectified neural signal to obtain an envelope of the rectified neural signal,
wherein sample values of the neural signal envelope represent neurological activity.

17. The method of claim 16, wherein the receiving further includes amplifying the received input signal.

18. The method of claim 16, wherein the filtering of the neural signal includes filtering by using a bandpass filter.

19. The method of claim 18, wherein the bandpass filter passes frequencies between 300 Hz and 10 kHz.

20. The method of claim 16, wherein integrating the filtered neural signal includes low pass filtering the neural signal.

21. The method of claim 16, further including sampling the integrated neural signal to obtain the sample values.

22. The method of claim 16, further including sampling the filtered neural to obtain the sampled values, such that the rectifying includes full-wave rectifying the sampled neural signal.

23. The method of claim 16, further including sampling the neural signal to obtain the sample values according to a low sampling rate.

24. The method of claim 23, wherein the sampling rate is less than about 2 kHz.

25. The method of claim 16, wherein the sampling includes converting the analog neural signal into a digital signal.

26. The method of claim 25, wherein the sampling rate is a low sampling rate.

27. The method of claim 26, wherein the sampling rate is less than about 2 kHz.

28. The method of claim 16, wherein the receiving further includes: receiving the input neural signal from an electrode in electrical contact with the cerebral cortex.

29. The method of claim 16, wherein the electrode is inserted into the cerebral cortex, and wherein the receiving further includes:
detecting a neural signals below a surface of the cerebral cortex.

30. The method of claim 16, wherein the electrode is part of a two-dimensional array of electrodes.
